(19) United States
(12) Patent Application Publication
(10) Pub. No.: US 2009/0281186 A1
(43) Pub. Date: Nov. 12, 2009

(54) Composition and Method for Reducing Stress

(76) Inventors: Melissa Lee, (US); Marvin A. Heuer, (US); Ken Clement, (US); Megan Thomas, (US)

Correspondence Address: IOVATE HEALTH SCIENCE RESEARCH INC., 381 North Service Road West, Oakville, ON, L6M 0H4, CA

(21) Appl. No.: 12/427,369

(22) Filed: Apr. 21, 2009

Related U.S. Application Data
(60) Provisional application No. 61/046,591, filed on Apr. 21, 2008.

(51) Int. Cl. A61K 31/195

(52) U.S. Cl. 514/563

(57) ABSTRACT

The present invention relates to a composition and method for reducing stress, in a mammal, wherein said composition acts by modulating levels and activities of various neurotransmitters and enhancing the phosphotidyl-inositol second messenger system affected by neurotransmitters. The composition of the present invention comprises at least L-theanine, including derivatives of L-theanine, and inositol, including derivatives of inositol.

### RELATED APPLICATIONS

The application is related to and claims benefit of priority to Applicant's U.S. Provisional Patent Application Ser. No. 61/046,591 entitled “Composition and Method for Reducing Stress,” filed Apr. 21, 2008, the disclosure of which is hereby fully incorporated by reference.

### FIELD OF THE INVENTION

The present invention relates to nutritional compositions and methods for reducing stress, in a mammal. Specifically, the present invention relates to compositions and methods comprising L-theanine or derivatives of L-theanine and inositol or derivatives of inositol. Where the ingredients act substantially simultaneously to modulate levels and activities of various neurotransmitters in the brain and enhance the phosphotidyl-inositol second messenger system commonly affected by neurotransmitters, particularly serotonin.

### BACKGROUND OF THE INVENTION

In the modern society, people experience various kinds of stress brought on by a myriad of causes. It has been reported that a variety of physical symptoms are caused by mental stress. For instance, it is recognized that mental stress has a negative influence on the circulatory system and immune system. However, the scientific concept and definition of stress have not yet been well established, so that means of evaluation of stress are not fully understood, nor have widely accepted methodologies been established. However, in recent years, more rigorous medical studies of stress have been attempted.

As an agent for preventing and mitigating mental and physical symptoms caused by stress, chemically synthesized medicaments such as a tranquilizer, an anti-anxiety agent, and sleeping pills are presently used. However, these medicaments have habituation and side effect problems, so that it is not preferable to use them daily for the purpose of preventing mental and physical symptoms caused by stress. Accordingly, an anti-stress agent that can be taken repeatedly and daily without any problems with safety, and that can mitigate and prevent mental and physical symptoms caused by stress are desired.

### SUMMARY OF THE INVENTION

The present invention relates to nutritional compositions and methods for reducing stress in a mammal. The nutritional compositions comprise at least an effective amount of L-theanine or derivatives of L-theanine and an effective amount of inositol or derivatives of inositol. The ingredients functioning substantially simultaneously to modulate levels and activities of various neurotransmitters in the brain and enhance the phosphotidyl-inositol second messenger system commonly affected by neurotransmitters, particularly serotonin. Both compositions and methods are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards nutritional compositions and methods for reducing stress in a mammal. The nutritional compositions comprise at least an effective amount L-theanine or derivatives of L-theanine and an effective amount of inositol or derivatives of inositol. The ingredients functioning substantially simultaneously to modulate levels and activities of various neurotransmitters in the brain and enhance the phosphotidyl-inositol second messenger system commonly affected by neurotransmitters, particularly serotonin.

As used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to a category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer to any substance that is a food, a part of food, or an extract of food which is suitable for consumption by an individual and providing physiological benefit which may be medical or health-related. Furthermore, the term has been used to refer to a product isolated, extracted or purified from foods or naturally-derived materials suitable for consumption by an individual and usually sold in medicinal forms, such as caplets, tablets, beads, powder, sachets, capsules, soft-gel™ caplets, gel-caps and the like, not associated with food.

Extracts suitable for use in the present invention may be produced by extraction methods as are known and accepted in the art such as alcoholic extraction, aqueous extractions, carbon dioxide extractions, for example.

As used herein, ‘L-theanine’ refers to the chemical 2-amino-4-(ethylcarbamoyl)butyric acid, (CAS Registry No. 3081-61-6), also known as N-ethyl-L-glutamine. Additionally, as used herein, ‘L-theanine’ also includes derivatives of L-theanine such as esters, amides, and salts, as well as other derivatives such as, for example, theanine ethyl ester, including derivatives having substantially similar pharmacoproperties to L-theanine upon metabolism to an active form.

As used herein, ‘inositol’ refers to the chemical cis-1,2,3,5-trans-4,6-cyclohexanehexol, (CAS Registry No. 87-89-8), also known as myo-inositol, meso-inositol, i-inositol, hexahydroxycyclohexane, cyclohexanehexol, cyclohexitol, meat sugar, inosite, mesoinosite, phaseomannite, or dambose. Additionally, as used herein, ‘inositol’ also includes derivatives of inositol such as esters and salts, as well as other derivatives such as, for example, 3-O-methyl-chiroinositol, a methoxy analogue of D-chiroinsitol, including derivatives having substantially similar pharmacoproperties to inositol upon metabolism to an active form.

As used herein the term ‘unmodified-release’ format is understood to be defined as pertaining to the dissolution and bioavailability profile of an ingested dietary ingredient wherein no additional modifications, be it chemical or physical, have been made to the ingredient in a naturally occurring form. It is also understood that unmodified-release is, essentially, immediate-release of active ingredients. This is further understood to be traditional- or conventional-release format where no slow-, delayed- or extended-release effect is incorporated.

As used here the term ‘controlled-release’ format is understood to be defined as a formulation of active ingredients and appropriate excipients in a specific format to facilitate a controlled- or non-immediate-release of active ingredients. The components of a controlled-release format may have been subjected to additional modifications, be it chemical or physical, with the specific intent to alter the dissolution or bioavailability profile from that of an ingredient in naturally occurring form.

As used herein the term ‘slow-release’ format is understood to be defined as a controlled-release format wherein the release of active ingredients are delayed for a period of time or gradually released over an extended period of time. This is accomplished through the use of specific excipients and may include structural features designed to facilitate controlled-release. It is further understood that a slow-release format releases active ingredients at a rate slower than immediate-release;

As used herein the term ‘delayed-release’ format is understood to be defined essentially as a controlled-release format wherein the components of the delayed-release format have undergone specific modifications, be it physical or chemical, to facilitate the release of active ingredients at a specific time after ingestion. It is further understood that delayed-release formats release active ingredients at a period of time later than unmodified release.

As used herein the term ‘quick-release’ format is understood to be defined essentially as unmodified release, as defined above. However, the term ‘quick-release’ may further include components having modifications, chemical or physical, to enhance the rate of dissolution or bioavailability of active ingredients.

### L-Theanine

L-Theanine is an amino acid that is commonly found in various teas, particularly Camellia sinensis. L-Theanine is a derivative of glutamine that is able to cross the blood-brain barrier, and as such has psychoactive properties. Although L-theanine is commonly found in green tea, it has activities which are distinct from the polyphenols and catechins that are typically associated with the beneficial effects of green tea. While catechins are generally associated with antioxidant activity, L-theanine is associated with anti-stress and cortisol control.

L-Theanine is thought to exert a relaxant effect by antagonizing the actions of glutamic acid in the brain. Glutamic acid is the most abundant excitatory neurotransmitter in the nervous system of mammals. Thus inhibition of glutamic acid binding results in decreased neuron excitation. Additionally, L-theanine administration, in rats, has been shown to increase dopamine release from the corpus straitum and increase serotonin levels in the hippocampus and thalamus. Therefore, the anti-stress effects of L-theanine are likely the result of modulation of levels and activities of various neurotransmitters.

In a double blind, placebo controlled, repeated measure design, sixteen individuals were tested under three treatments in order to compare the acute effects of L-theanine versus traditionally prescribed bezodiazepam on induced anxiety. These treatments included; placebo, 200 mg of L-theanine and 1 mg of alprazolam. Acute administration of L-theanine caused a tranquil state in subjects during the experimental relaxed state while alprazolam had no effect.

In a double blind, counterbalanced study, twelve individuals underwent four different trials; one in which they were administered 200 mg of L-theanine before the induced anxiety task; one in which they were administered 200 mg of L-theanine midway through the induced anxiety task; and two where they were given a placebo or nothing. Psychological measures revealed a greater increase in the perception of stress in the placebo condition compared to the other conditions. Cardiovascular measures demonstrated that during the stress task heart rates were higher in the placebo condition compared to the L-theanine groups. Overall, subjects showed more activation of the sympathetic nervous system during an acute stress period under the placebo condition then any of the other three conditions. Therefore, acute stress responses were reduced by oral administration of L-theanine, as indicated by both psychological and physiological measures.

It is herein understood by the inventors that inclusion of L-theanine or derivatives of L-theanine in a nutritional composition, will act to modulate levels and activities of various neurotransmitters in the brain. L-theanine or derivatives of L-theanine antagonize the actions of the excitatory neurotransmitter, glutamic acid, thereby reducing excitation of neurons, while at the same increasing levels of inhibitory neurotransmitters such as dopamine and serotonin.

In an embodiment of the present invention which is set forth in greater detail in the examples below, the nutritional composition comprises an effective amount of L-theanine or derivatives of L-theanine. A serving of the composition comprises from about 0.005 g to about 2.0 g of L-theanine or derivatives of L-theanine. In a preferred dosage of the present invention, a serving of the nutritional composition comprises about 0.025 g to about 0.50 g of L-theanine or derivatives of L-theanine. In a more preferred dosage of the present invention, a serving of the nutritional composition comprises about 0.05 g to about 0.030 g of L-theanine or derivatives of L-theanine. In the preferred dosage of the present invention, a serving of the nutritional composition comprises about 0.20 g of L-theanine or derivatives of L-theanine.

### Inositol

Inositol is a simple, naturally occurring isomer of glucose. It is often considered to be a member of the B vitamin family and is present in most plant sources in the form of the fiber component, phytic acid. Inositol is present in animals in the form of myo-inositol, which interestingly is most abundant in the central nervous system. Inositol plays an important role in signal transduction as it is a key intermediate of a secondary messenger system that is commonly used by various neurotransmitters.

In a double-blind, controlled, random-order crossover study, the effects of inositol and the drug fluvoxamine for treatment of panic disorders were compared. Inositol and fluvoxamine were shown to be equally effective in treating panic disorders. However, symptoms of nausea and dizziness were significantly more prevalent in those individuals receiving fluvoxamine as compared to those receiving inositol.

Additionally, in a double-blind, placebo-controlled cross-over trial 21 patients with panic disorder were given 12 grams of inositol per day for a period of four weeks. The severity and frequency of panic attacks were significantly decreased with inositol administration compared to placebo. It has been proposed that inositol is similar, in mechanism, to many antidepressant drugs, particularly serotonin reuptake inhibitors. Key serotonin receptors are linked to the phosphotidyl-inositol second messenger system, therefore increased levels of inositol could result in increased propagation of second messenger signals.

It is herein understood by the inventors that inclusion of inositol or derivatives of inositol in a nutritional composition, will act to attenuate stress. Inositol or derivatives of inositol play a major role as the structural basis for a number of secondary messengers. Thus, exogenous administration of inositol or derivatives of inositol will enhance the phosphotidyl-inositol second messenger system commonly affected by neurotransmitters, particularly serotonin.

In an embodiment of the present invention which is set forth in greater detail in the examples below, the nutritional composition comprises an effective amount of inositol or derivatives of inositol. A serving of the composition comprises from about 0.0001 g to about 10 g of inositol or derivatives of inositol. In a preferred dosage of the present invention, a serving of the nutritional composition comprises about 0.10 g to about 8.0 g of inositol or derivatives of inositol. In a more preferred dosage of the present invention, a serving of the nutritional composition comprises about 2.0 g to about 6.0 g of inositol or derivatives of inositol. In the preferred dosage of the present invention, a serving of the nutritional composition comprises about 4.0 g of inositol or derivatives of inositol.

In various embodiments of the present invention, which are set forth in detail in examples below, the nutritional composition of the present invention comprises at least L-theanine or derivatives of L-theanine and inositol or derivatives of inositol. The nutritional composition is provided in any acceptable and suitable oral dosage form as known in the art. Reduced excitation of the sympathetic nervous system by antagonizing glutamine receptors and increasing levels of inhibitory neurotransmitters; and enhancement of the phosphotidyl-inositol second messenger system commonly affected by neurotransmitters, are induced and carried out in an individual by administration of the composition of the present invention.

The nutritional composition of the present invention may be administered in a dosage form having controlled release characteristics, e.g. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the nutritional composition may be administered in the form of a multi-compartment capsule which combines both immediate release and time-release characteristics. Individual components of the nutritional composition may be contained in differential compartments of such a capsule such that the specific components may be released rapidly while others are time-dependently released. Alternatively, a uniform mixture of the various components of the present invention may be divided into both immediate release and time-release compartments to provide a multi-phasic release profile.

Embodiments of the present invention of the present invention having multi-phasic release profiles may do so according the methods disclosed in U.S. patent application Ser. No. 11/709,525 entitled “Method for a Supplemental Dietary Composition Having a Multi-Phase Dissolution Profile” filed Feb. 21, 2007, which is herein fully incorporated by reference. The aforementioned discloses a method of providing a multi-phasic dissolution profile through the use of differentially-sized milled particles.

The present invention comprises L-theanine or derivatives of L-theanine, which have been shown to act to decrease psychological and physiological perceptions of stress. This reduction in stress results from L-theanine's reduction in the binding of glutamic acid to glutamine receptors in neurons, resulting in decreased stimulation of the sympathetic nervous system, and increased levels of dopamine and serotonin.

Additionally, the present invention comprises inositol or derivatives of inositol that have been shown to reduce stress, in mammals. Exogenous administration of inositol or derivatives of inositol will enhance the phosphotidyl-inositol second messenger system commonly affected by neurotransmitters, particularly serotonin.

Furthermore, it is herein understood by the inventors that the components of the present invention will act in concert through at least the aforementioned, distinct mechanisms to reduce stress, in a mammal.

Additional embodiments of the present invention may also include portions of the composition as fine-milled ingredients. U.S. patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

According to various embodiments of the present invention, the nutritional composition may be consumed in any form. For instance, the dosage form of the nutritional composition may be provided as, e.g. a powder beverage mix, a liquid beverage, a ready-to-eat bar or drink product, a capsule, a liquid capsule, a tablet, a caplet, sachet, or as a dietary gel. The preferred dosage forms of the present invention are provided as a caplet or as a liquid capsule.

In addition to the foregoing, compositions of the present invention include formulations further comprising additional active ingredients and/or inactive ingredients, including solvents, diluents, suspension aids, thickening or emulsifying agents, sweeteners, flavorings, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and which may also be suitable for use in formulations of the present invention. Except insofar as any conventional carrier medium is incompatible with the ingredients of the invention, such as by producing any undesirable effect or otherwise interacting in a deleterious manner with any other ingredient(s) of the formulation, its use is contemplated to be within the scope of this invention.

Furthermore, the dosage form of the nutritional composition may be provided in accordance with customary processing techniques for herbal and nutritional compositions in any of the forms mentioned above. Additionally, the nutritional composition set forth in the example embodiment herein disclosed may contain any appropriate number and type of excipients, as is well known in the art. By way of ingestion of the composition of the present invention, a method for reducing stress, in a mammal, is provided. The method of the present invention comprises at least the step of administering to an individual an effective amount of the composition of the present invention.

Although the following examples illustrate the practice of the present invention in two of its embodiments however the examples should not be construed as limiting the scope of the invention. Other embodiments will be readily apparent to one of skill in the art from consideration of the specifications and examples.

### EXAMPLES

### Example 1

A stress reducing composition comprising the following ingredients per serving:
about 0.20 g of L-theanine, and about 0.35 g of inositol;
which is prepared for consumption as two caplets to be taken three times daily.

### Example 2

A stress reducing composition comprising the following ingredients per serving:
about 0.20 g of L-theanine, and about 4.0 g of inositol;
which is prepared for consumption as a powder, contained in sachets, to be taken three times daily.

### Example 3

A stress reducing composition comprising the following ingredients per serving:
about 0.20 g of L-theanine, and about 4.0 g of inositol;
which is prepared for consumption as four caplets to be taken three times daily.

### Extensions And Alternatives

In the foregoing specification, the invention has been described with a specific embodiment thereof; however, it will be evident that various modifications and changes may be made thereto without departing from the broader spirit and scope of the invention.

What is claimed:

1. A stress reducing composition comprising:
L-theanine; and
inositol.

2. The stress reducing composition of claim 1, wherein the amount of the L-theanine is from about 0.005 g to about 0.20 g, per serving; and the amount of inositol is from about 0.0001 g to about 6.0 g, per serving.

3. The stress reducing composition of claim 1, wherein the amount of L-theanine is about 0.20 g, per serving; and the amount of inositol is about 4.0 g, per serving.

4. The stress reducing composition of claim 1, wherein the stress reducing composition is provided to a mammal in need thereof in an acceptable oral dosage format.

5. The stress reducing composition of claim 4, wherein the acceptable oral dosage format is selected from the group consisting of tablets, caplets, beads, powder, capsules and soft-gel capsules.

6. The stress reducing composition of claim 5, wherein the acceptable oral dosage format is provided in an unmodified release form.

7. The stress reducing composition of claim 5, wherein the acceptable oral dosage format is provided in a time-release form.

8. The stress reducing composition of claim 5, wherein the acceptable oral dosage form consists of an unmodified release in combination with a time-release.

9. The stress reducing compositions of claim 7, wherein the time-release formats are selected from the group consisting of controlled-release, slow release, delayed-release and quick-release.

10. The stress reducing composition of claim 8, wherein the time-release formats are selected from the group consisting of controlled-release, slow-release, delayed-release and quick-release.

11. A method for reducing stress in a mammal comprising:
administering to said mammal a composition comprising:
L-theanine; and
inositol.

12. The method of claim 11, wherein the amount of the L-theanine is from about 0.005 g to about 0.20 g, per serving; and the amount of inositol is from about 0.0001 g to about 6.0 g, per serving.

13. The method of claim 11, wherein the amount of L-theanine or is about 0.20 g, per serving; and the amount of inositol is about 4.0 g, per serving.

14. The method of claim 11, wherein the stress reducing composition is provided to a mammal in need thereof in an acceptable oral dosage format.

15. The method of claim 14, wherein the acceptable oral dosage format is selected from the group consisting of tablets, caplets, beads, powder, capsules and soft-gel capsules.

16. The method of claim 14, wherein the acceptable oral dosage format is provided in an unmodified release form.

17. The method of claim 14, wherein the acceptable oral dosage format is provided in a time-release form.

18. The method of claim 15, wherein the acceptable oral dosage form consists of an unmodified release in combination with a time-release.

19. The method of claim 17, wherein the time-release formats are selected from the group consisting of controlled-release, slow release, delayed-release and quick-release.

20. The method of claim 18, wherein the time-release formats are selected from the group consisting of controlled-release, slow-release, delayed-release and quick-release.
